(19) United States
(12) Patent Application Publication
(10) Pub. No.: US 2005/0160701 A1
(43) Pub. Date: Jul. 28, 2005

(54) Tissue preservation assembly and method of making and using the same

(76) Inventor: Timothy A. Stevens, Warwick, NY (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(21) Appl. No.: 11/017,479

(22) Filed: Dec. 20, 2004

Related U.S. Application Data
(60) Provisional application No. 60/532,767, filed on Dec. 24, 2003.

(51) Int. Cl. B65B 55/02; A61L 2/08

(52) U.S. Cl. 53/425; 422/22; 435/284.1; 435/307.1

(57) ABSTRACT

A sterilized device for storing and transferring a tissue sample collected in a sterile field is provided having a hermetically sealed package containing a tissue storage chamber sealed with a removable cap creating a sample storage chamber housing a tissue preservation agent. The package may be provided as a lid sealed to a tray, a plastic bag that is sealed, or two pieces of plastic sealed along the edges. An interior compartment is created within the package, which is sterile along with the sample container and its contents. A tissue preservation agent is provided to stabilized RNA in a tissue sample. The sterilized device is intended for use in storing and transferring a tissue sample from a patient while in a sterile field. The sterilized device is a single assembly that eliminates disrupting a sterile field in an operating room and transferring an exposed tissue sample out of the sterile field.

### CROSS-REFERENCE TO RELATED APPLICATION

This application claims the benefit of U.S. Provisional Application No. 60/532,767, filed Dec. 24, 2003.

### BACKGROUND OF THE INVENTION

1. Field of the Invention

The present invention relates to a sterile tissue preservation assembly. More particularly, the present invention relates to a sterilized device for storing and transferring a tissue sample from a sterile field.

2. Description of Related Art

Tissue storage chambers are used in the process of retrieving and collecting a biological sample for the purpose of storing a preserved tissue sample. More particularly, the tissue storage chamber provides an environment for containing a ribonucleic acid (RNA) preservation agent that preserves and stabilizes the RNA component of a tissue sample. During the process of tissue sample collection, the preservation and stabilization of RNA within the tissue sample is vital to prevent RNA degradation prior to molecular analysis of the tissue sample. Isolating intact RNA is required for quantitative and qualitative analysis of RNA expression by Northern Blot hybridization, nuclease protection assays and reverse transcriptase polymerase chain reaction.

RNA can be degraded through enzymatic activity and bio-contamination. In order to minimize the activity of RNase liberated during cellular lysis from collected samples such as intact tissues or cells high in endogenous ribonuclease, cells must be quickly homogenized in a powerful protein denaturant such as guanidinium isothiocyanate. Alternatively, intact tissues or cells must be rapidly frozen in liquid nitrogen at <80° C., stored, ground into a powder, and finally homogenized in a RNA extraction buffer when ready for RNA extraction.

Recently, new methods to preserve RNA in cell and tissue samples were disclosed in U.S. Pat. No. 6,204,375, which has been assigned to Ambion, Inc. The RNA preservation described in U.S. Pat. No. 6,204,375 is marketed by Ambion, Inc. and Qiagen under the trademarks RNAlater™ and RNeasy®, respectively, both sold in non-sterile form. The RNAlater™ and RNeasy® products, both solutions of ammonium sulfate, have provided researchers and doctors the ability to store tissue or cell samples for one day at 37° C., for one week at room temperature, for one month at 4° C., and indefinitely at minus 20° C. or 80° C. The growing use of RNAlater™ and RNeasy® have improved the ability of pathologists to preserve RNA in the hospital setting to some extent, by providing a less labor-intensive method for preserving RNA by submerging a tissue sample in RNAlater™ or RNeasy®.

Such ammonium sulfate preservation solutions are currently packaged in relatively large (five cc) Nalgene containers or vials. In practice, the surgeon or pathologist transfers the solution from the Nalgene container to a secondary container prior to placing a piece of cellular tissue or blood sample into the secondary container to begin RNA preservation. However, the step of transferring the solution conflicts with operating room procedures relating to sterility. Specifically, maintaining a sterile field and preventing the introduction of pathogenic microorganisms in the blood is one of the most serious concerns in performing surgery. A sterile field is the aseptic area created around a procedure site where only sterile items are allowed. If one non-sterile object or person enters the sterile field, the field is no longer considered sterile.

The use of ammonium sulfate in current practice in containers in an operating room situation is inadequate for maintaining a sterile field. Accordingly, the collection of a tissue sample in an operating room typically occurs by way of retrieving a tissue specimen from a patient, placing the tissue specimen on a sterile gauze pad moistened with sterile saline and transporting the tissue specimen to a container which is separately filled with RNA preservative from a large supply container. This process is time consuming, and exposes RNA within the tissue sample to the risk of enzymatic degradation in transit and possibly bio-contamination if exposed to contaminated air outside of the sterile field. Clearly, improvements are needed and desired for the steps of collecting a tissue sample and preserving the RNA in the tissue sample, all within the sterile field of the operating room.

Accordingly, there is a present need for a sterilized device capable of entering the sterile field of an operating room without contamination, which is used for storing and preserving a tissue sample collected in a sterile field.

### SUMMARY OF THE INVENTION

Generally, the present invention is directed to a sterilized device, including a hermetically sealed package containing a container sealed with a removable cap and having a tissue storage chamber containing a tissue preservation agent therein. The present invention is an improvement over the prior art as it provides a sterilized container containing a tissue preservation agent sealed in a package with a sterile interior. The present invention is a further improvement over the prior art as it provides a sterilized container capable of entering the sterile field of an operating room without disrupting the sterility of the field. The present invention is an even further improvement over the prior art as it eliminates the need for collecting a tissue sample from a patient in a sterile field, placing the tissue sample on a sterile transfer mechanism and transporting the tissue sample outside of the sterile field for preservation of the tissue and storage in a storage container.

Specifically, the present invention is a sterilized device comprising a sterilized container sealed with a removable cap that creates a tissue storage chamber containing a tissue preservation agent, whereby the sterilized container is hermetically sealed within a package having a sterile interior. Preferably, the container includes a container with an integrally formed cap. Preferably, the tissue preservation agent is a salt including sulfate salts or any other salt capable of preserving the RNA in a tissue or cell sample.

Desirably, the package for the container includes a top lid sheet that is hermetically sealed to a bottom tray after the container having a tissue preservation agent therein is placed within the bottom tray. Alternatively, the package for the container may include a plastic bag, which contains the container, and is hermetically sealed at the open end. A further alternative of the package includes two separate, perimetrically-cut, flat pieces of plastic that are hermetically sealed to create an interior compartment or pouch that houses the container with the tissue preservation agent therein.

Additionally, the present invention involves a method of manufacturing the aforementioned sterilized device. Specifically, the present invention is a method of manufacturing an assembly including the steps of providing a tissue storage chamber, depositing a tissue preservation agent in the tissue storage chamber, closing the tissue storage chamber to maintain the reagent therein, sealing the tissue storage chamber within a container package, and sterilizing the container package containing the tissue storage chamber. Preferably, the step of sterilizing the device includes irradiation of the device.

### BRIEF DESCRIPTION OF THE DRAWINGS

FIG. 1 is a perspective view of a tissue storage chamber with a cap removably attached thereto in accordance with the present invention;

FIG. 2 is a longitudinal cross-section taken along line 2-2 of FIG. 1;

FIG. 3 is a perspective view of a tissue storage chamber which is sealed within a package having a sterile interior in accordance with the present invention;

FIG. 4 is an exploded view of a tissue storage chamber sealed within a package having a sterile interior as shown in FIG. 3;

FIG. 5 is a cross-section of the tissue storage chamber sealed within a package having a sterile interior as shown in FIG. 3;

FIG. 6 is a perspective view of an alternate embodiment of the present invention including a tissue storage chamber sealed within a bag-like package having a sterile interior;

FIG. 7 is an exploded perspective view of the tissue storage chamber and bag-like package having a sterile interior as shown in FIG. 6; and

FIG. 8 is a partial cross-section view of the tissue storage chamber sealed within a bag-like package having a sterile interior as shown in FIG. 6.

### DETAILED DESCRIPTION OF THE INVENTION

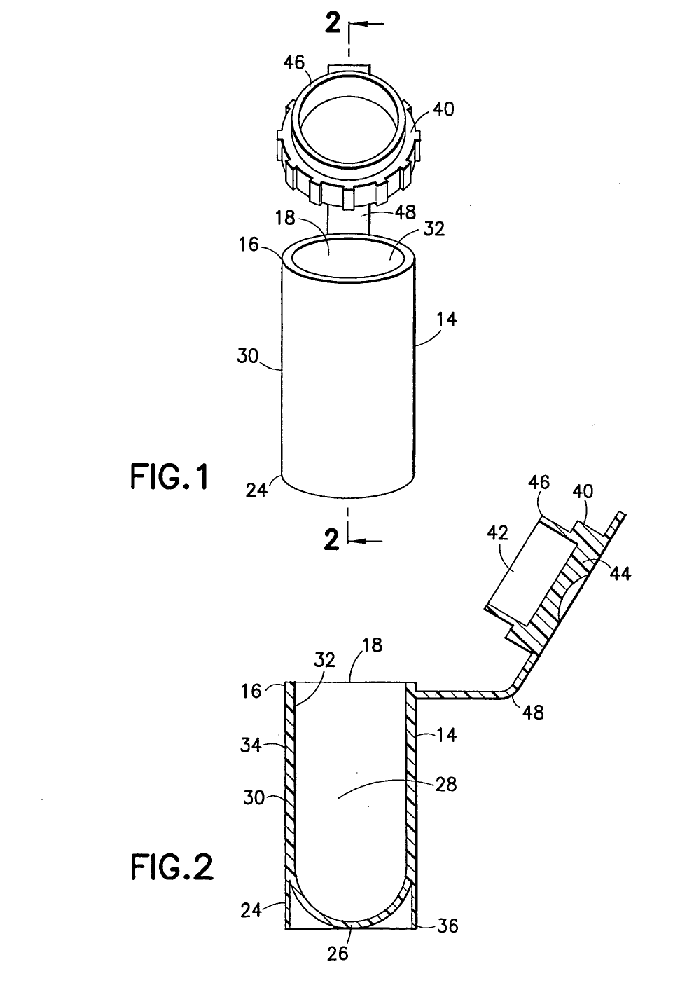

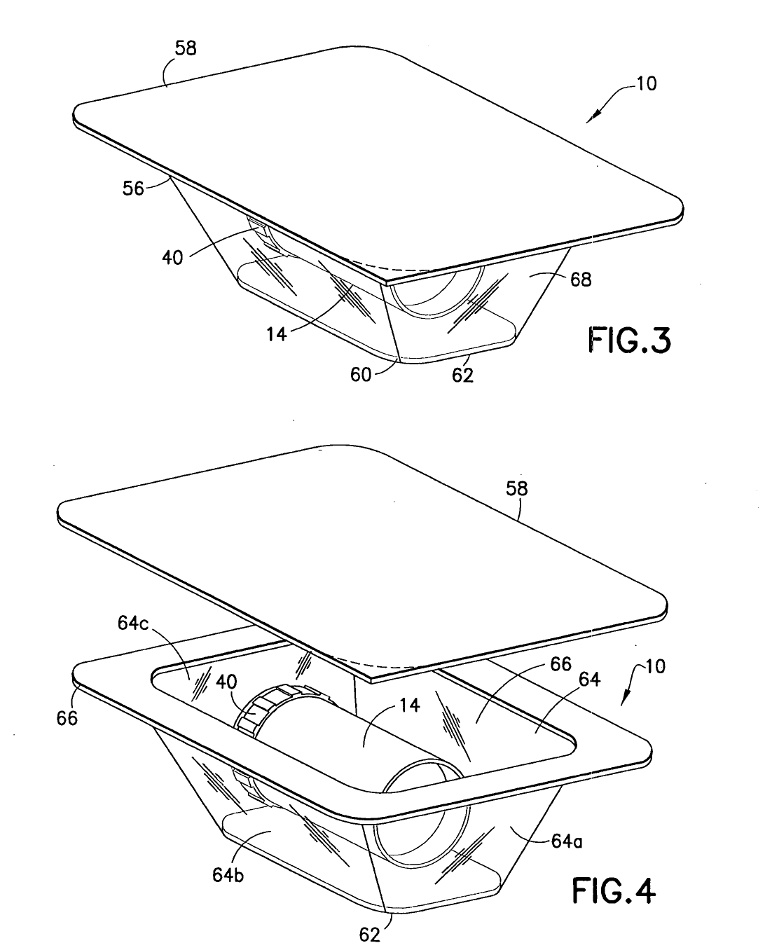

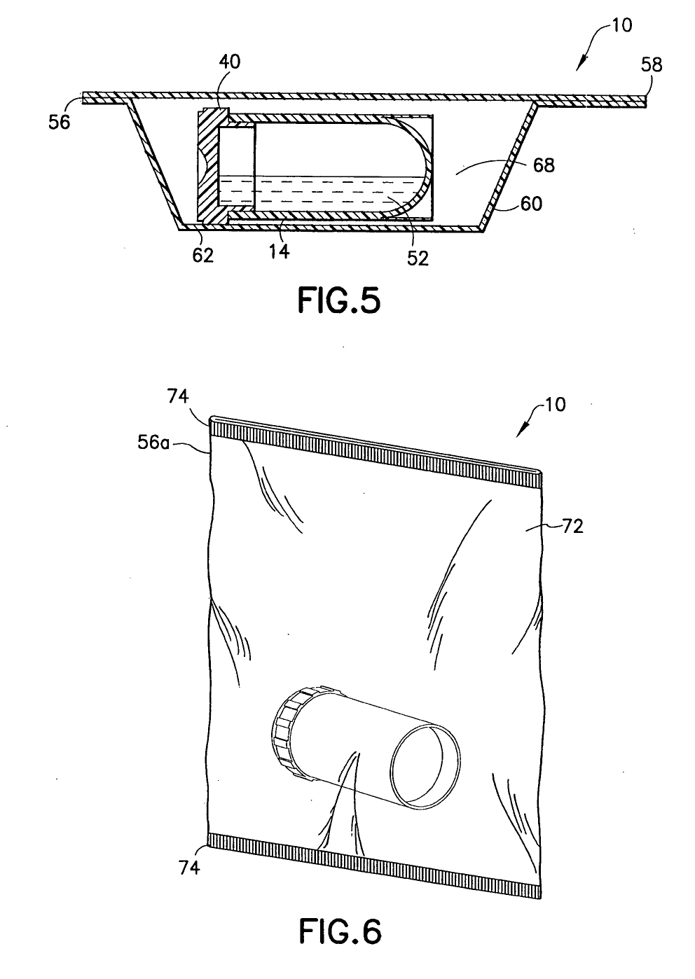

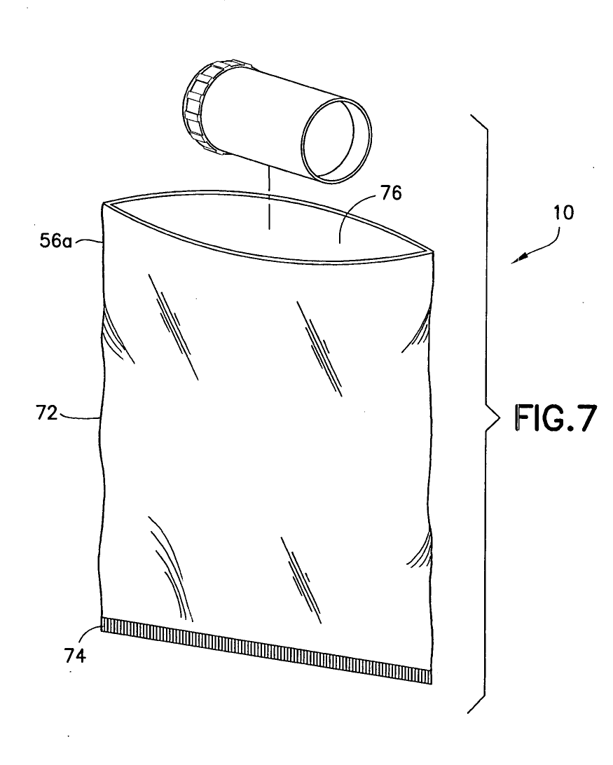

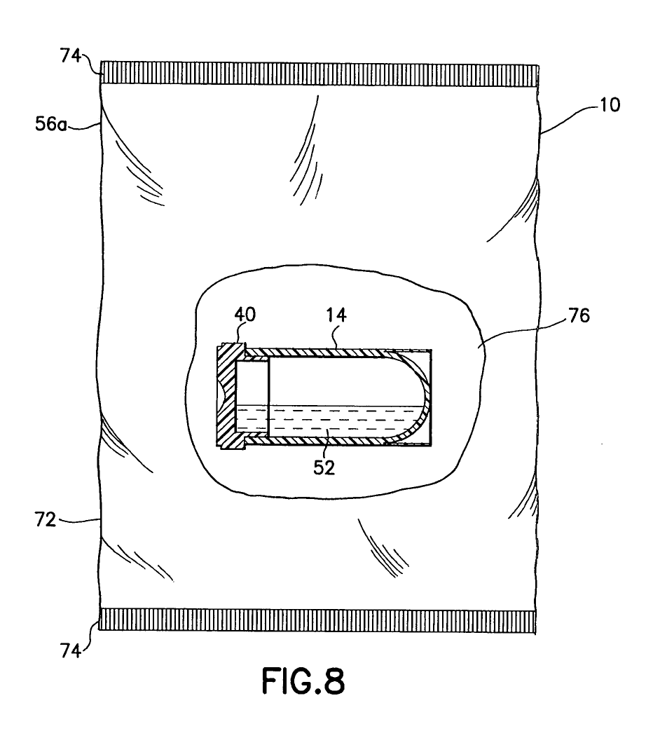

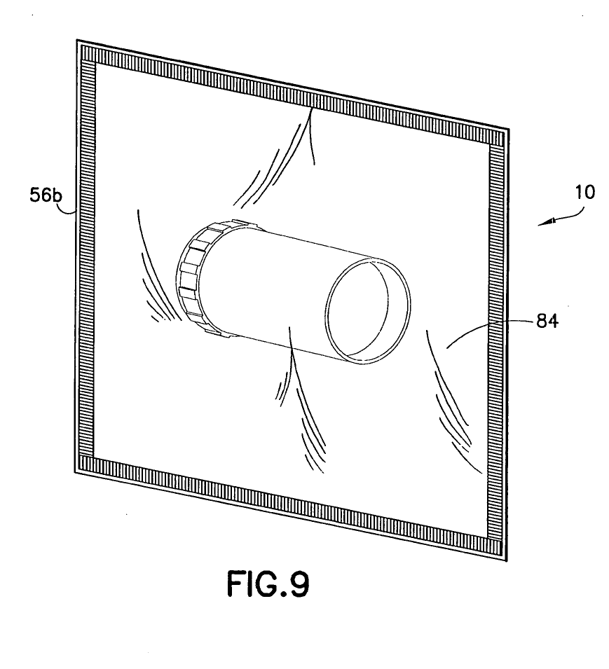

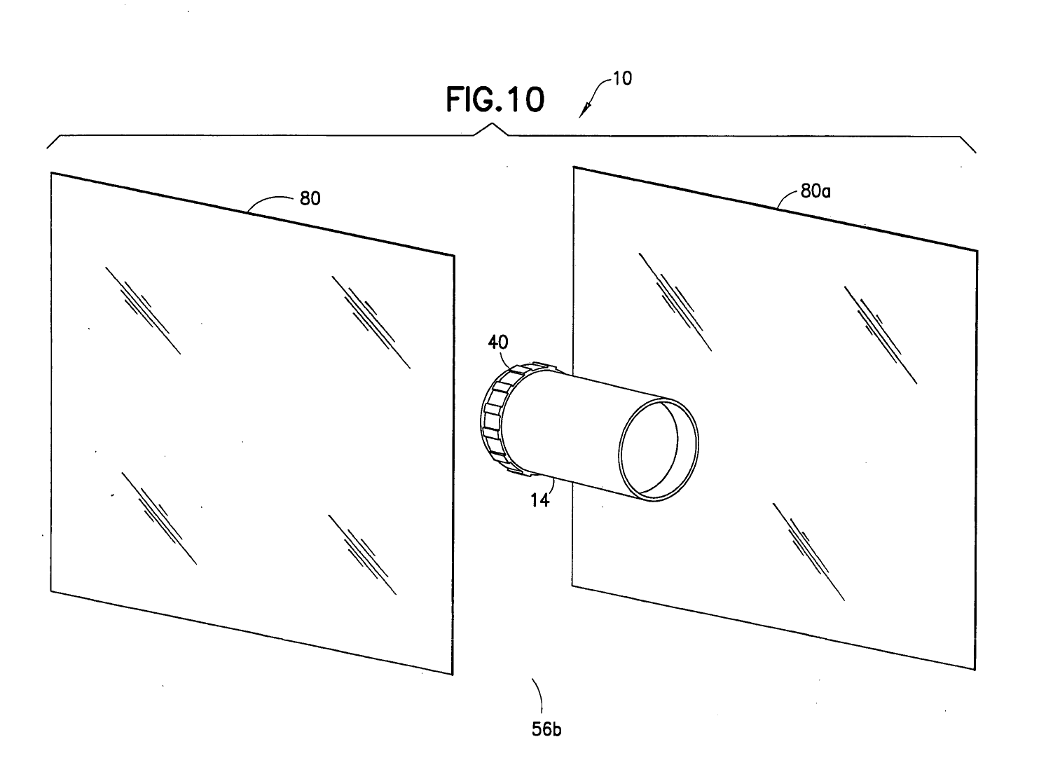

Referring to the drawings in which like reference characters refer to like parts throughout the several views thereof, FIGS. 1 through 8 illustrate embodiments of a sterilized device 10 for storing and transferring tissue sample collected in a sterile field comprised of tissue storage chamber 14 with removable cap 40 containing tissue preservation agent 52, all hermetically sealed within a package such as package 56, 56a, or 56b. The present invention is generally described in terms of sterilized device 10 with several embodiments.

An embodiment of tissue storage chamber 14 as shown specifically in FIGS. 1-2 and throughout remaining FIGS. 3-8 generally comprises cap 40 that is capable of being removably sealed to tissue storage chamber 14. Generally, tissue storage chamber 14 has upper portion 16, lower portion 24 and sidewall 30 extending therebetween. Sidewall 30 defines inner surface 32 and outer surface 34. Upper portion 16 includes open end 18, and lower portion 24 comprises closed bottom 26, defining tissue storage chamber 28 therebetween. Annular skirt 36 extends from sidewall 30 in a direction away from upper portion 16 towards lower portion 24. Annular skirt 36 provides structure for allowing tissue storage chamber 14 to be placed and maintained upright on a flat surface.

Cap 40 for tissue storage chamber 14 has open end 42, closed end 44 and sidewall 46 extending therebetween. Cap 40 and tissue storage chamber 14 mate between upper portion 16 of tissue storage chamber 14 and sidewall 46 of cap 40 to secure and seal cap 40 to tissue storage chamber 14 to provide a leak resistant assembly. This may be accomplished by a friction fit, a threaded engagement, a locking cam with lugs or other means common in the art. Strap 48 is provided to connect cap 40 to tissue storage chamber 14. Strap 48 can be integrally formed with either one of or both cap 40 and tissue storage chamber 14, desirably forming a unitary structure with a connection web therebetween. The present invention can incorporate one of many embodiments of a tissue storage chamber which may be known in the art.

Tissue preservation agent 52 is contained within tissue storage chamber 28. Tissue preservation agent 52 may be any medium that is capable of stabilizing and preserving RNA in a collected tissue or cell sample when that sample is submersed in tissue preservation agent 52. Preferably, tissue preservation agent 52 comprises a sulfate salt solution. Alternatively, tissue preservation agent 52 comprises a solution including a preservative selected from the group consisting of ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate, zinc sulfate, methanol, trichloroacetic acid, 1-propanol, 2-propanol, polyethylene glycol, and acetic acid. Tissue preservation agents prepared in accordance with U.S. Pat. No. 6,204,375, incorporated herein by reference thereto, are useful within the scope of the present invention. Tissue preservation agent 52 is comprised of a solution that contains effective amounts of a preservative capable of stabilizing and preserving RNA in a tissue or cell sample for subsequent molecular analysis.

Particular embodiments of sterile device 10 including tissue storage chamber 14 having cap 40 sealed to tissue storage chamber 14 with tissue preservation agent 52 therein sealed in package 56, 56a, 56b present are shown in FIGS. 3 through 8. Preferably, package 56 is comprised of top lid sheet 58 hermetically sealed to bottom tray 60 to create interior compartment 68 as shown in FIGS. 3 through 5. Top lid sheet 58 comprises a piece of flat plastic or any other material capable of maintaining an airtight seal when sealed to bottom tray 60. Bottom tray 60, a three-dimensional structure, is comprised of bottom wall 62, four sidewalls 64, 64a, 64b, 64c and open top 66. Bottom tray 60 is comprised of a plastic material or any other material capable of maintaining an airtight seal when sealed to top lid sheet 58.

Alternatively, package 56a is comprised of polyethylene or polypropylene bag 72 as shown in FIGS. 6 through 8. Bag 72 has hermetically sealed edges 74 thereby creating interior compartment 76 that houses tissue storage chamber 14 having cap 40 sealed to tissue storage chamber 14 with tissue preservation agent 52 therein.

In a further alternative embodiment as shown in FIGS. 9-10, package 56b comprises two pieces of generally planar sheet material 80, 80a constructed from a polymeric material such as polyethylene or polypropylene. Such plastic sheet material 80, 80a may be cut around a perimetrical edge in a desired shape, such as a square, and positioned to sandwich tissue storage chamber 14, and hermetically sealed along all four edges to create interior compartment 84.

In any of the aforementioned embodiments of package 56, 56a, and 56b, sterile interior compartment 68, 76, and 84, respectively, contains sterile tissue storage chamber 14 containing sterile tissue preservation agent 52. This sterility provides the means for tissue storage chamber 14 to be removed from package 56, 56a, 56b and transported into the sterile field of the operating room. Alternatively, the sterility provides the means for sterile device 10 to be resterilized immediately prior to entry into the sterile field of the operating room. In order to meet sterility requirements for the operating room, sterile interior compartment 68, 76, 84 of package 56, 56a, 56b, respectively, has preferably a sterility assurance level of better than one in one thousand. More preferably, sterile interior compartment 68, 76, 84 of package 56, 56a, 56b has a sterility assurance level of better that one in one million.

The present invention is also directed to a method of manufacturing the aforementioned embodiments of sterile device 10. The method comprises the steps of providing tissue storage chamber 14, depositing into tissue storage chamber 14 tissue preservation agent 52, closing tissue storage chamber 14 to maintain tissue preservation agent 52 therein with an airtight seal, sealing tissue storage chamber 14 within container package 56, 56a, 56b, and sterilizing container package 56, 56a, 56b with tissue storage chamber 14 sealed therein. The result of the method of manufacture is sterile device 10 having sterile interior compartment 68, 76, 84 containing sterile tissue storage chamber 14 containing sterile tissue preservation agent 52.

Tissue preservation agent 52 is included within tissue storage chamber 14 in amounts effective for RNA stabilization that are known in the art. An adequate amount of tissue preservation agent 52 is placed in tissue storage chamber 14 to fully submerse any tissue or cell sample placed within tissue storage chamber 14. After tissue preservation agent 52 is placed within tissue storage chamber 14, tissue storage chamber 14 is closed by removably sealing cap 40 to tissue storage chamber 14 thereby creating an airtight seal. For example, the airtight seal may be created by a friction fit, a threaded engagement, a locking cam with lugs or other means or structure common in the art, which is based upon the type of tissue storage chamber 14 employed.

In the present invention, tissue storage chamber 14 with tissue preservation agent 52 sealed therein, is sealed within package 56, 56a, 56b. Package 56, 56a, 56b is comprised of any one of the packages described and claimed herein, namely one having top lid 58 sealed to bottom tray 60, bag 72 with the opening sealed, or two square cut pieces of plastic 80, 80a sealed at all four edges. In a preferred embodiment, top lid 58 is hermetically and removably sealed to bottom tray 60 by heat sealing top lid 58 to bottom tray 60. An adhesive may also be placed between top lid 58 and bottom tray 60 to seal the two components together. In another preferred embodiment, the open end of bag 72 is hermetically sealed. The sealing may occur by heat sealing an edge 74 of bag 72. An adhesive may also be incorporated to facilitate the sealing of bag 72. In a further embodiment, two square-cut pieces of plastic 80, 80a are hermetically sealed together. The sealing may occur by heat sealing all four edges of the pieces of plastic 80, 80a. An adhesive may also be incorporated to facilitate the sealing of the pieces of plastic 80, 80a. In any of the preferred embodiments, once sealed, interior compartment 68, 76, 84 is created in package 56, 56a, 56b, respectively, that houses sealed tissue storage chamber 14 having tissue preservation agent 52 therein.

A further step of sterilizing device 10 may be provided in the method of manufacture, which comprises irradiating device 10 comprising package 56, 56a, 56b with tissue storage chamber 14 containing tissue preservation agent 52 sealed therein. Preferably, the sterilization step comprises subjecting device 10 to Cobalt-60 radiation process, as is known in the art. An effective amount of Cobalt-60 radiation is in the amount of 2.5 to 100 kGy. Alternatively, the sterilization process comprises subjecting the device 10 to electron beam irradiation, or other acceptable sterilization processes that may be known in the art. In any type of sterilization used, the resulting sterilized device 10 possesses interior compartment 68, 76, 84 with a sterility assurance level of better than one in a thousand and, preferably, better than one in one million.

While the present invention is satisfied by embodiments in many different forms, there is shown in the drawings and described herein in detail the preferred embodiments of the invention, with the understanding that the present disclosure is to be considered as exemplary of the principles of the invention and is not intended to limit the invention to the embodiments illustrated. Various other embodiments will be apparent to and readily made by those skilled in the art without departing from the scope and spirit of the invention. The scope of the invention will be measured by the appended claims and their equivalents.

1. A sterilized device for storing and transferring a tissue sample collected in a sterile field, the sterilized device comprising a container with a cap removably attached to an open end of the container creating a tissue storage chamber having a tissue preservation agent therein, the container sealed within a package, the package containing a sterile interior.

2. The device of claim 1, wherein the sterile interior of the package has a sterility assurance level of better than one in one thousand.

3. The device of claim 1, wherein said sterile interior of the package has a sterility assurance level of better than one in one million.

4. The device of claim 1, wherein the package comprises a pouch comprised of polyethylene or polypropylene.

5. The device of claim 1, wherein the package comprises a top lid sheet hermetically sealed with a bottom tray.

6. The device of claim 1, wherein the tissue preservation agent is a salt.

7. The device of claim 6, wherein the salt is a sulfate salt.

8. The device of claim 6, wherein the salt is a salt selected from the group consisting of ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate, zinc sulfate, methanol, trichloroacetic acid, 1-propanol, 2-propanol, polyethylene glycol and acetic acid.

9. The device of claim 1, wherein the container and the cap are integrally formed.

10. The device of claim 1, wherein the cap is connected to the container through a connection web.

11. A method for manufacturing an assembly comprising the steps of:
providing a tissue storage chamber;
depositing into the tissue storage chamber a tissue preservation agent;
closing the tissue storage chamber to maintain the tissue preservation agent therein;
sealing the tissue storage chamber within a container package; and
sterilizing the container package with the tissue storage chamber sealed therein.

12. The method of claim 11, wherein the sterilization step comprises irradiating the container package.

13. The method of claim 12, wherein the sterilization step comprises subjecting the container package to a Cobalt-60 radiation process.

14. The method of claim 13, wherein the container package is subjected to Cobalt-60 radiation in the amount of 2.5-100 kGy.

15. The method of claim 11, wherein the sterilization step comprises subjecting the container package to an electron beam process.

16. The method of claim 11, wherein the closing step comprises mating the tissue storage chamber with a removable cap.

17. The method of claim 16, wherein the cap comprises an elastomeric sealing element.

18. The method of claim 11, wherein the tissue preservation agent comprises a salt.

19. The method of claim 18, wherein the salt is a sulfate salt.

20. The method of claim 18, wherein the salt is selected from the group consisting of ammonium sulfate, ammonium bisulfate, cesium sulfate, cadmium sulfate, cesium iron (II) sulfate, chromium (III) sulfate, cobalt (II) sulfate, copper (II) sulfate, lithium sulfate, magnesium sulfate, manganese sulfate, potassium sulfate, sodium sulfate, and zinc sulfate.

21. The method of claim 11, wherein the container package comprises a pouch comprised of polyethylene or polypropylene.

22. The method of claim 21, wherein the package comprises a top sheet and a bottom tray.

23. The method of claim 22, wherein the sealing step comprising placing the tissue storage chamber within the bottom tray and hermetically sealing the top sheet to the bottom tray.

24. A kit for containing and preserving a tissue sample collected within a sterile environment, the kit comprising a container contained within a sterilized package, the container including a cap removably attached to an open end of the container creating a tissue storage chamber having a tissue preservation agent therein, the container sealed within a sterile interior of the sterilized package.
